(19) United States
(12) Patent Application Publication
(10) Pub. No.: US 2017/0216628 A1
(43) Pub. Date: Aug. 3, 2017

(54) METHODS AND DEVICES FOR STEREOTACTIC RADIOSURGERY

(71) Applicants: University Hospital Ostrava, Ostrava-Poruba (CZ); E & H Services, A.S., Praha (CZ)

(72) Inventors: Jakub Cvek, Frydek-Mistek (CZ); Tomás Ocelka, Sedliste ve Slezsku (CZ)

(21) Appl. No.: 15/014,767

(22) Filed: Feb. 3, 2016

(51) Int. Cl. A61N 5/10

(52) U.S. Cl. CPC A61N 5/1067; A61N 5/1049; A61N 5/1083; A61N 2005/1051; A61N 2005/1061

(57) ABSTRACT

Methods, devices, and systems can be used to improve accuracy and precision of stereotactic radiosurgery. For example, this document provides methods and materials for using a fiducial marker device for stereotactic radiosurgery of ocular disorders. The fiducial marker device of the invention enables positional tracking of the target tissue during the stereotactic radiosurgery procedure despite occasional movement of the eye being subjected to stereotactic radiosurgery.

### BACKGROUND

1. Technical Field

This document relates to methods and devices for improving stereotactic radiosurgery of cancer tissue in or on an eye, and for reducing the potential for injury of tissue adjacent to the cancer tissue. For example, this document relates to treating cancer tissue from a freely movable eye of a mammal by stereotactic radiosurgery using a contact lens comprising radiopaque fiducials.

2. Background Information

Stereotactic radiosurgery (SRS) utilizes externally generated ionizing radiation to inactivate or eradicate defined targets without the need of a surgical incision. Accurate delivery of the radiation to target tissue requires setting up a reliable spatial frame of reference. SRS employs a three-dimensional coordinate system for localization of the target tissue inside the body. In some cases, bone landmarks, which are known to have a constant spatial relation to the target tissue, can provide such a reliable frame of reference. If such bone landmarks are not available within the required proximity, markings (fiducials) can be used for positional tracking of the target tissue during the SRS procedure.

Using SRS for the treatment of ocular disorders is particularly challenging because the eye is not visible on planar radiographs, and because the eye can move during the SRS procedure. Even after anesthesia, the oculomotor muscles can continue to cause movements of the eyeball and thus limit the accuracy of the SRS procedure. Therefore, different eye immobilization techniques are known in the art. Such techniques include passive immobilization systems which restrict ocular movements, and active immobilization systems wherein the patient controls the eye position by fixating on a light source, for example.

### SUMMARY

This document provides methods, devices, and systems for improving the accuracy of stereotactic radiosurgery in or on an eye while reducing injury to adjacent tissue. For example, this document provides a method of treating cancer tissue within a freely movable eye of a mammal by stereotactic radiosurgery using a contact lens comprising radiopaque fiducials. As described further below, the fiducial marker devices provided herein facilitate positional tracking of the target tissue during the SRS procedure. Accordingly, occasional movement(s) of the eye during the SRS procedure can be adjusted for. That is, accurate directionally targeted delivery of the radiation can be made throughout the SRS procedure even when the eye moves during the procedure.

As described herein, mammals suffering from an ocular disorder can be treated with SRS using the fiducial marker devices provided herein. For example, the fiducial marker devices can be placed on the eye of a human patient undergoing SRS to constantly track the position of the target tissue within the eye. In some cases, the target tissue can be intraocular or periocular tumor tissue. In these cases, the fiducial marker device can help to improve accuracy and precision of the ablation of the tumor tissue. In some cases, the fiducial marker device can help to reduce radiation damage to the tissue surrounding or adjacent to the target tissue.

In some cases, the fiducial marker device can comprise fiducials arranged on a base substrate. For example, radiopaque grains can be used as fiducials and placed on a base lens, which are can be configured to fit onto a mammal's eyeball. In some cases, the fiducials can be made of radiopaque materials, e.g., gold, platinum, zirconium, or an alloy of gold and tin. In some cases, the base lens can be a rigid contact lens (or analog thereto), combining a shape for comfortable wear on the eyeball with spatial stability and durability. In some cases, the lens can be measured to fit onto the eyeball of a dog, a rodent, a cat, a monkey, or a human. Size, material and arrangement of the fiducials on the lens facilitate the detection of movements (e.g., translational and rotational) of the eye.

As described herein, the fiducial marker device can be used for positional tracking of the eyeball of a mammal during SRS of a variety of ocular disorders including, but not limited to, glaucoma, age-related macular degeneration, idiopathic orbital inflammatory disease and eye tumors. Tumors of the eye that can be tracked with the devices and methods provided herein include, but are not limited to, uveal melanoma, optic nerve sheath meningioma, and ocular lymphoma.

In one implementation, a method of treating cancer tissue of a movable eye of a mammal is provided. During the method, the eye can be in direct contact with a contact lens comprising two or more fiducials. The method includes: (a) receiving imaging data of at least a portion of the eye and the two or more fiducials; (b) identifying a location of the cancer tissue based on the imaging data; (c) determining a spatial relationship between the location and the two or more fiducials; (d) delivering radiation to the eye to treat the cancer tissue; and (e) while the step (d) is being performed, receiving additional imaging data of the two or more fiducials and redirecting the radiation delivery based on the additional imaging data.

Such a method of treating cancer tissue of a movable eye of a mammal may optionally include one or more of the following features. The redirecting may be in response to detecting a movement of the eye during the delivery of the radiation. The method may also include providing, based on the spatial relationship between the location and the two or more fiducials, information about a new location of the cancer tissue to an actuator that automatically repositions a radiation source from which the radiation is delivered. The contact lens may comprise a rigid contact lens. The contact lens may include at least three fiducials. The contact lens may include four fiducials. The two or more fiducials may include a radiopaque material. The radiopaque material may be selected from the group consisting of gold, platinum, zirconium, and an alloy of gold and tin. The radiopaque material may be an alloy of gold and tin. In some implementations, the mammal is a human. The cancer tissue may be cancer tissue pertaining to the group consisting of uveal melanoma, optic nerve sheath meningioma, and ocular lymphoma.

In another implementation, a fiducial marker device provided herein includes a contact lens and two or more fiducials attached to the contact lens. The two or more fiducials may include a radiopaque material. A ratio of an outer diameter of the contact lens to a base diameter of one fiducial of the two or more fiducials can be in a range of about 5:1 to about 10:1.

Such a fiducial marker device may optionally include one or more of the following features. An outer diameter of the contact lens may be in a range from about 13 mm to about 16 mm. An outer diameter of a base portion of one fiducial of the two or more fiducials is in a range from 1.6 mm to 2.5 mm. The two or more fiducials may include at least three fiducials. The two or more fiducials may include four fiducials. The four fiducials may be separated by equal 90 degree angular increments. An outer diameter of a base portion of one fiducial of the two or more fiducials may be in a range from 1.8 mm to 2.2 mm. The fiducials may be positioned near an outer rim of the contact lens. The two or more fiducials may be hemispherically shaped. Each hemispherically shaped fiducial of the two or more fiducials may have a volume of at least 1.2 mm³. The contact lens may be a rigid contact lens. The radiopaque material may be selected from the group consisting of gold, platinum, zirconium, and an alloy of gold and tin. The radiopaque material may be an alloy of gold and tin.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF DRAWINGS

FIGS. 1 and 2 are side views of an example contact lens that includes fiducials in accordance with some embodiments provided herein.

FIGS. 3 and 4 are side views of another example contact lens that includes fiducials in accordance with some embodiments provided herein.

FIGS. 5 and 6 are side views of another example contact lens that includes fiducials in accordance with some embodiments provided herein.

FIGS. 7 and 8 are side views of another example contact lens that includes fiducials in accordance with some embodiments provided herein.

FIG. 9 is a photographic plan view of contact lens that includes fiducials placed on a hypothetical aim.

FIG. 10 shows storage of a contact lens including fiducials placed in a vessel filled with preservation solution.

FIG. 11 is a flowchart of an example method for treating cancer tissue in or on an eye using a contact lens that includes fiducials, in accordance with some embodiments provided herein.

Like reference numbers represent corresponding parts throughout.

### DETAILED DESCRIPTION

This document provides methods, devices, and systems for improving the performance of stereotactic radiosurgery on an eye, while reducing the potential for injury to adjacent tissue. For example, this document provides a method of stereotactic radiosurgery to treat cancer tissue within a freely movable eye of a mammal by using a contact lens comprising radiopaque fiducials. As described further below, the fiducial marker devices provided herein facilitate positional tracking of the target tissue during the SRS procedure. Accordingly, occasional movement(s) of the eye during the SRS procedure can be positionally adjusted for. That is, accurate directionally targeted delivery of the radiation can be made throughout the SRS procedure even when the eye moves during the procedure.

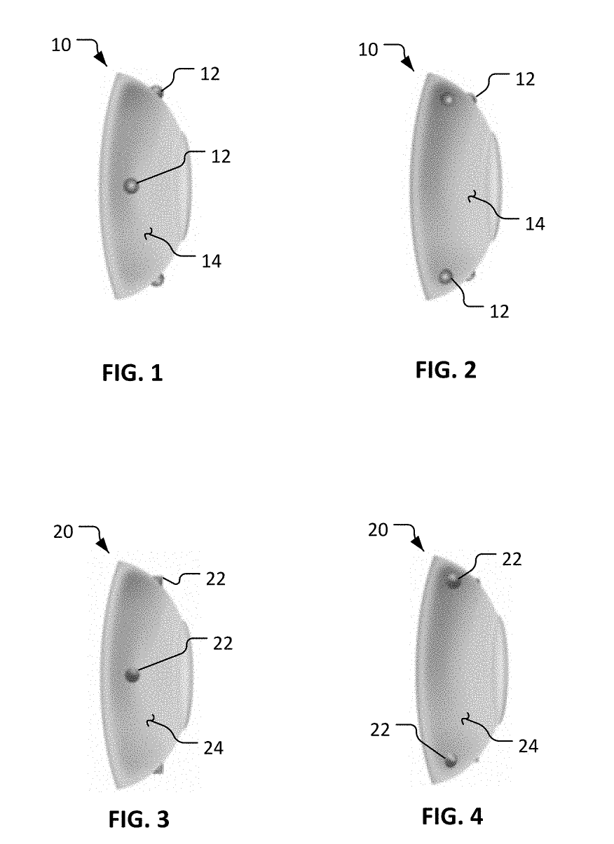

Referring to FIGS. 1 and 2, a fiducial marker device 10 can be used to enhance positional tracking of the target tissue during an SRS procedure. Fiducial marker device 10 includes a base member 14 and a plurality of fiducials 12 mounted onto the base member 14.

In the depicted embodiment, four fiducial members 12 are mounted to base member 14. In some embodiments, fewer than or more than four fiducial members 12 are mounted to base member 14. For example, in some embodiments two, three, four, five, six, seven, eight, nine, ten, or more than ten fiducial members 12 are mounted to base member 14.

Fiducial members 12 can be made of any suitable material(s) that are detectable by X-ray radiation (i.e., radiopaque materials). Suitable materials include, but are not limited to, gold, platinum, zirconium, and an alloy of gold and tin.

Base member 14 can be made of any material(s) that are suitable for a contact lens. In some embodiments, rigid, soft, or hybrid contact lenses can be used as base member 14. Both, soft and rigid contact lenses may provide certain advantages as base member 14. For example, rigid contact lenses can be especially resistant to spatial deformation. Also, rigid contact lenses can be more amenable for modifications, such as the fixation of fiducial members 12 thereon. Soft contact lenses can be resilient when subjected to modifications, such as the fixation of fiducial members 12 thereon. The size of base member 14 can be chosen to fit the particular eyeball to be treated.

Base members 14 having a wide range of optical powers can be used for fiducial marker device 10 as provided herein. Knowledge of the optical power of base member 14 is used to direct the radiation beam of the SRS system directionally accurately to the location of the target tissue.

In some embodiments, base member 14 has the complete shape of a typical contact lens. Alternatively, in some embodiments base member 14 includes one or more fenestrations, which can be beneficial for providing moisture to the eye in some cases.

In some embodiments, a polymer or other type of binder material is used to attach fiducial members 12 to base member 14. In some embodiments, the outer surfaces of fiducial members 12 are well polished, whereas, in some embodiments, inner surface area (i.e., the contact surface with base member 14) is microscopically roughened to enhance adhesion with base member 14.

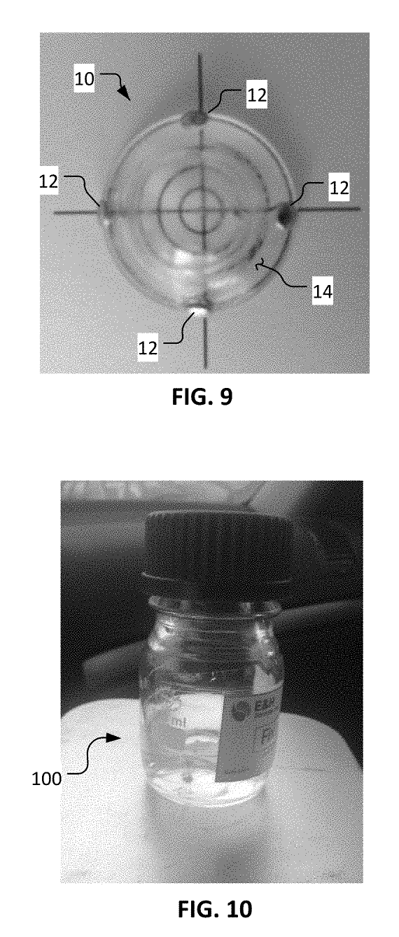

Referring also to FIG. 9, fiducial members 12 can be attached to base member 14 in a symmetrical arrangement. For example, in the depicted embodiment that includes four fiducial members 12, each fiducial members 12 is located at a 90° angle in relation to adjacent fiducial members 12. That is, the four fiducial members 12 are spaced apart from each other by equal angular increments of approximately 90°. Moreover, each fiducial member 12 is equidistant from the center of base member 14, and positioned near to the outer rim of base member 14.

Still referring to FIGS. 1 and 2, fiducial members 12 can be manufactured into a variety of different sizes and shapes. For example, in the depicted embodiment fiducial members 12 are hemispherical and fiducial members 12 have a base diameter sized in a range from about 1.8 mm to about 2.2 mm. In some embodiments, larger or smaller fiducial members 12 can be used. For example, in some embodiments fiducial members 12 have a base diameter sized in a range from about 1.0 mm to about 3.0 mm, or from about 1.6 mm to 2.5 mm. In some embodiments, the dimensions of fiducial members 14 are defined so that fiducial members 12 have a volume of at least 1.5 mm³, or at least 1.2 mm³.

In some embodiments, base member 14 has an outer diameter in a range from about 13 mm to about 16 mm, or from about 10 mm to about 20 mm. Base member 14 is scalable to accommodate any eye size.

In some embodiments, the ratio of the outer diameter of base member 14 to the base diameter of fiducial members 14 is in the range of about 5:1 to about 10:1.

In some embodiments, the ratio of the outer diameter of base member 14 to the base diameter of fiducial members 14 is in the range of about 3:1 to about 12:1, or about 5:1 to about 7:1, or about 6:1 to about 8:1, or about 8:1 to about 10:1.

Referring to FIGS. 3 and 4, a fiducial marker device 20 can be used to enhance positional tracking of the target tissue during an SRS procedure. Fiducial marker device 20 includes a base member 24 and a plurality of fiducials 22 mounted onto the base member 24.

In the depicted embodiment, four fiducial members 22 are mounted to base member 24. In some embodiments, fewer than or more than four fiducial members 22 are mounted to base member 24. For example, in some embodiments two, three, four, five, six, seven, eight, nine, ten, or more than ten fiducial members 22 are mounted to base member 24.

Fiducial members 22 and base member 24 can be made of any material(s) and made by any of the processes described above in reference to fiducial marker device 10.

In some embodiments, such as the depicted embodiment, fiducial members 22 are conically shaped members. In some embodiments, fiducial members 22 are frustoconical in shape.

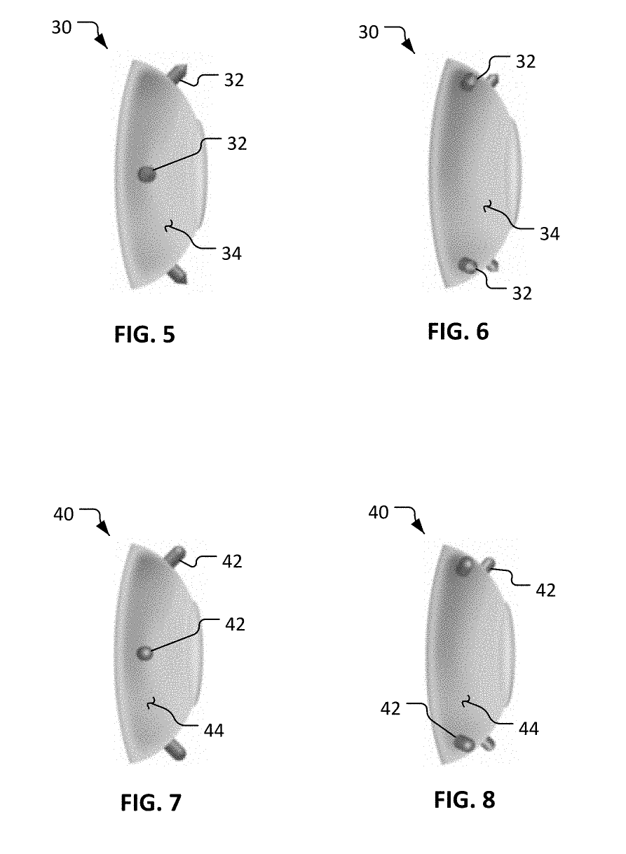

Referring to FIGS. 5 and 6, a fiducial marker device 30 can be used to enhance positional tracking of the target tissue during an SRS procedure. Fiducial marker device 30 includes a base member 34 and a plurality of fiducials 32 mounted onto the base member 34.

In the depicted embodiment, four fiducial members 32 are mounted to base member 34. In some embodiments, fewer than or more than four fiducial members 32 are mounted to base member 34. For example, in some embodiments two, three, four, five, six, seven, eight, nine, ten, or more than ten fiducial members 32 are mounted to base member 34.

Fiducial members 32 and base member 34 can be made of any material(s) and made by any of the processes described above in reference to fiducial marker device 10.

In some embodiments, such as the depicted embodiment, fiducial members 32 are columnar (cylindrical) with conically-shaped tips. In some embodiments, the tips of fiducial members 32 are frustoconical in shape.

Referring to FIGS. 7 and 8, a fiducial marker device 40 can be used to enhance positional tracking of the target tissue during an SRS procedure. Fiducial marker device 40 includes a base member 44 and a plurality of fiducials 42 mounted onto the base member 44.

In the depicted embodiment, four fiducial members 42 are mounted to base member 44. In some embodiments, fewer than or more than four fiducial members 42 are mounted to base member 44. For example, in some embodiments two, three, four, five, six, seven, eight, nine, ten, or more than ten fiducial members 42 are mounted to base member 44.

Fiducial members 42 and base member 44 can be made of any material(s) and made by any of the processes described above in reference to fiducial marker device 10.

In some embodiments, such as the depicted embodiment, fiducial members 42 are columnar (cylindrical) with hemispherically-shaped tips. In some embodiments, the tips of fiducial members 42 are pyramidal in shape.

Referring to FIG. 10, the fiducial marker devices provided herein can be stored and preserved within a liquid-tight vessel 100 that also contains a preservative solution. Such an arrangement facilitates storing of the fiducial marker device before and/or between applications. Any solution appropriate for storing and/or cleaning of contact lenses can be used, e.g. a saline solution.

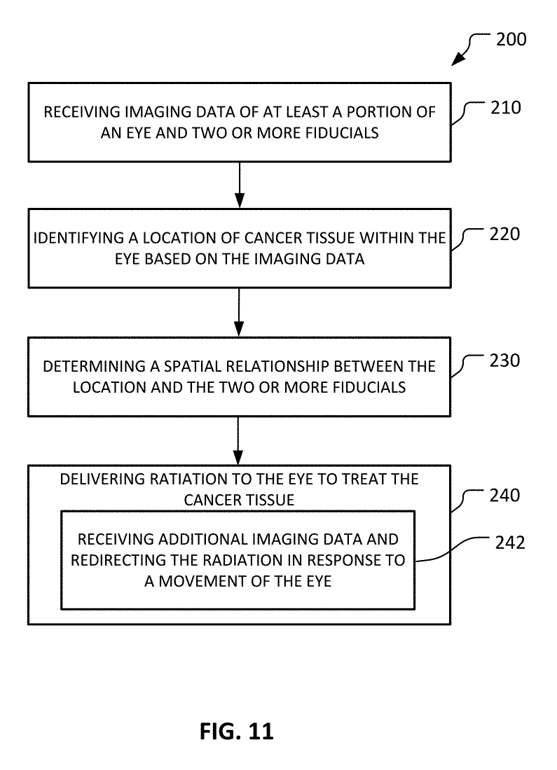

Referring to FIG. 11, a method 200 can be used to treat cancer tissue within or on a movable eye of a mammal (e.g., human beings and other mammalians). Method 200 includes the use of a fiducial marker device (e.g., a fiducial marker device 10, 20, 30, or 40, or the like) as described herein. That is, the eye receiving the treatment is in direct contact with a contact lens that includes two or more fiducials.

In general, method 200 is a stereotactic radiosurgery (SRS) technique. Such SRS techniques may include the use of a computer-controlled robot that can slowly move around the patient to the various locations from which it delivers radiation to the targeted tissue (e.g., tumor). In some cases, method 200 may be performed using a system such as, but not limited to, a CyberKnife® Robotic Radiosurgery System from Accuray® Incorporated.

Accurate delivery of the radiation to target tissue requires setting up a reliable spatial frame of reference. SRS employs a three-dimensional coordinate system for localization of the target tissue inside the body.

In some cases, the eye receiving treatment can move during the SRS procedure. Even after anesthesia, the oculomotor muscles can continue to cause movements of the eyeball. Such movement can tend to limit the accuracy of the SRS procedure. Method 200 is advantageous in that it includes aspects that can compensate for eye movements. That is, as described further below, occasional movement(s) of the eye during the SRS procedure can be positionally adjusted for. In result, accurate directionally targeted delivery of the radiation can be made throughout the SRS procedure, even when the eye moves during the procedure.

At operation 210, a computer-controller of the stereotactic radiosurgery system receives imaging data. The imaging data includes images of at least a portion of the eye undergoing the treatment. Additionally, the imaging data includes images of the two or more fiducials of the fiducial marker device that is engaged on the eye undergoing the treatment.

At operation 220, the computer-controller of the stereotactic radiosurgery system is used to identify a location of the cancer tissue within (or on) the eye, based on the imaging data received in operation 210. That is, the three-dimensional location in space of the cancer tissue is identified.

The three-dimensional location in space of each of the two or more fiducials of the fiducial marker device that is engaged on the eye undergoing the treatment can also be identified by the computer-controller of the stereotactic radiosurgery system.

At operation 230, the computer-controller of the stereotactic radiosurgery system determines a spatial relationship between the location of the cancer tissue and the locations of the two or more fiducials. The fiducial marker device that includes the two or more fiducials is generally stationary in relation to the eye. As the eye moves, the fiducial marker device also moves in correspondence to the eye. Hence, the spatial relationship between the location of the cancer tissue within (or on) the eye and the locations of the two or more fiducials remains substantially constant even though the eye may move.

Operation 240 includes using the computer-controller of the stereotactic radiosurgery system to deliver radiation directed to the cancer tissue within (or on) the eye, while operation 242 is contemporaneously performed. At operation 242, while radiation is being delivered to the cancer tissue, additional imaging data of the two or more fiducials is received, and, in response to at least one movement of the eye, the radiation delivery is redirected (based on the additional imaging data).

As explained in detail elsewhere herein, the eye may move during the radiation delivery portion of an SRS procedure. Operation 240 (which includes operation 242) advantageously compensates for such movement(s). That is, the computer-controller of the stereotactic radiosurgery system uses the determined spatial relationships between the location of the cancer tissue and the locations of the two or more fiducials to dynamically adjust (redirect) the aim of the radiation delivery in response to eye movements. Hence, accurate directionally targeted delivery of the radiation can be made throughout the SRS procedure, even when the eye moves during the procedure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method of treating cancer tissue of a movable eye of a mammal, wherein said eye is in direct contact with a contact lens comprising two or more fiducials, said method comprising:
(a) receiving imaging data of at least a portion of said eye and said two or more fiducials;
(b) identifying a location of said cancer tissue based on said imaging data;
(c) determining a spatial relationship between said location and said two or more fiducials;
(d) delivering radiation to said eye to treat said cancer tissue; and
(e) while said step (d) is being performed, receiving additional imaging data of said two or more fiducials and redirecting said radiation delivery based on said additional imaging data.

2. The method of claim 1, wherein said redirecting is in response to detecting a movement of said eye during said delivery of said radiation.

3. The method of claim 1, further comprising providing, based on said spatial relationship between said location and said two or more fiducials, information about a new location of said cancer tissue to an actuator that automatically repositions a radiation source from which said radiation is delivered.

4. The method of claim 1, wherein said contact lens comprises a rigid contact lens.

5. The method of claim 1, wherein said contact lens comprises at least three fiducials.

6. The method of claim 5, wherein said contact lens comprises four fiducials.

7. The method of claim 1, wherein said two or more fiducials comprise a radiopaque material.

8. The method of claim 7, wherein said radiopaque material is selected from the group consisting of gold, platinum, zirconium, and an alloy of gold and tin.

9. The method of claim 8, wherein said radiopaque material is an alloy of gold and tin.

10. The method of claim 1, wherein said mammal is a human.

11. The method of claim 1, wherein said cancer tissue is cancer tissue pertaining to the group consisting of uveal melanoma, optic nerve sheath meningioma, and ocular lymphoma.

12. A fiducial marker device comprising:
a contact lens; and
two or more fiducials attached to said contact lens,
wherein said two or more fiducials comprise a radiopaque material, and wherein a ratio of an outer diameter of said contact lens to a base diameter of one fiducial of the two or more fiducials is in a range of about 5:1 to about 10:1.

13. The fiducial marker device of claim 12, wherein an outer diameter of said contact lens is in a range from about 13 mm to about 16 mm.

14. The fiducial marker device of claim 12, wherein an outer diameter of a base portion of one fiducial of said two or more fiducials is in a range from 1.6 mm to 2.5 mm.

15. The fiducial marker device of claim 12, wherein said two or more fiducials comprises at least three fiducials.

16. The fiducial marker device of claim 15, wherein said two or more fiducials comprises four fiducials.

17. The fiducial marker device of claim 16, wherein said four fiducials are separated by equal 90 degree angular increments.

18. The fiducial marker device of claim 17, wherein an outer diameter of a base portion of one fiducial of said two or more fiducials is in a range from 1.8 mm to 2.2 mm.

19. The fiducial marker device of claim 16, wherein the fiducials are positioned near an outer rim of said contact lens.

20. The fiducial marker device of claim 12, wherein said two or more fiducials are hemispherically shaped.

21. The fiducial marker device of claim 20, wherein said each hemispherically shaped fiducial of said two or more fiducials has a volume of at least 1.2 mm³.

22. The fiducial marker device of claim 12, wherein said contact lens is a rigid contact lens.

23. The device of claim 12, wherein said radiopaque material is selected from the group consisting of gold, platinum, zirconium, and an alloy of gold and tin.

24. The device of claim 23, wherein said radiopaque material is an alloy of gold and tin.
